(19) United States
(12) Patent Application Publication
(10) Pub. No.: US 2007/0060916 A1
(43) Pub. Date: Mar. 15, 2007

(54) System and network for remote medical procedures

(76) Inventor: Carlo Pappone, Milano (IT)

Correspondence Address: Edward Renner, Suite 400, 7700 Bonhomme, St. Louis, MO, 63105, US

(21) Appl. No.: 11/478,440

(22) Filed: Jun. 29, 2006

Related U.S. Application Data
(60) Provisional application No. 60/702,488, filed on Jul. 26, 2005.

(51) Int. Cl. A61B 17/00

(52) U.S. Cl. 606/1

(57) ABSTRACT

A system is provided for enabling remote monitoring of a medical procedure being performed on a patient. The system comprises at least one full operator station having a navigation control system for controlling the orientation of a minimally interventional medical device that is to be guided within a subject body's anatomy, and one or more remote operator stations in communication with the at least one full operator station, wherein the medical procedure may be monitored from the one or more remote operator stations. The remote operator station may be a visitor operator station, a passive operator station, an active operator station, or another full operator station. The remote operator stations provide for educational training, hands on training through remotely performing procedures in a limited capacity, and full control of a medical procedure from a location that is remote from the patient and facility where the procedure is being conducted.

### CROSS-REFERENCE TO RELATED APPLICATIONS

This application claims the benefit of U.S. Provisional Patent Application Ser. No. 60/702,488, filed Jul. 26, 2005, the entire disclosure of which is incorporated herein by reference.

### FIELD OF THE INVENTION

The present invention relates to the medical procedures which utilize navigation of medical devices within a subject body, and more specifically to remotely performing medical procedures utilizing navigation of medical devices in a subject's body.

### BACKGROUND OF THE INVENTION

Navigation systems have recently been commercially developed for actuation of medical devices to be steered within a patient's anatomy, from a remote location nearby the patient. An example is the Niobe magnetic navigation system developed and sold by Stereotaxis, Inc. Such a system typically allows for control of the navigation of a minimally interventional device with the help of a Graphical User Interface and user input devices such as a mouse, keyboard, joystick or other form of interface input device.

### SUMMARY OF THE INVENTION

The present invention relates to a system and network for remotely performing various medical procedures. Preferably, the system comprises equipment for performing medical procedures using minimally interventional devices that are navigated through a subject's body. In one embodiment in accordance with the present invention, a network and system are provided for enabling remote actuation of a minimally interventional medical device that is to be guided within a subject body's anatomy, for the purpose of performing various medical procedures. The system comprises a navigation system for controlling the orientation of a medical device

In accordance with one aspect of the present invention, a system and network is provided for enabling remote monitoring of a medical procedure being performed in a patient's body. The system comprises at least one full operator station having a navigation control system for controlling the orientation of a minimally interventional medical device that is to be guided within a subject body's anatomy, and one or more remote operator stations in communication with the at least one full operator station, wherein the medical procedure may be monitored from the one or more remote operator stations. The remote operator station may be a visitor operator station, a passive operator station, an active operator station, or another full operator station.

In another aspect of the present invention, a system is provided for enabling an operator to remotely perform a medical procedure in a patient's body at a remote location. The system comprises at least one full operator station having a navigation control system for controlling the orientation of a minimally interventional medical device that is to be guided within a subject body's anatomy, one or more remote operator stations in communication with the at least one full operator station, wherein the medical procedure may be controlled at least partially by an operator at the one or more remote operator stations. Accordingly, a system and network of operator stations may be provided that provides for both educational training, hands on training through remotely performing procedures in a limited capacity, and full control of a medical procedure from a remote location that may be a great distance from the patient and medical facility where the procedure is being conducted.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an illustration of one embodiment of a system and network for enabling control of minimally interventional medical devices from a remote location to perform various medical procedures;

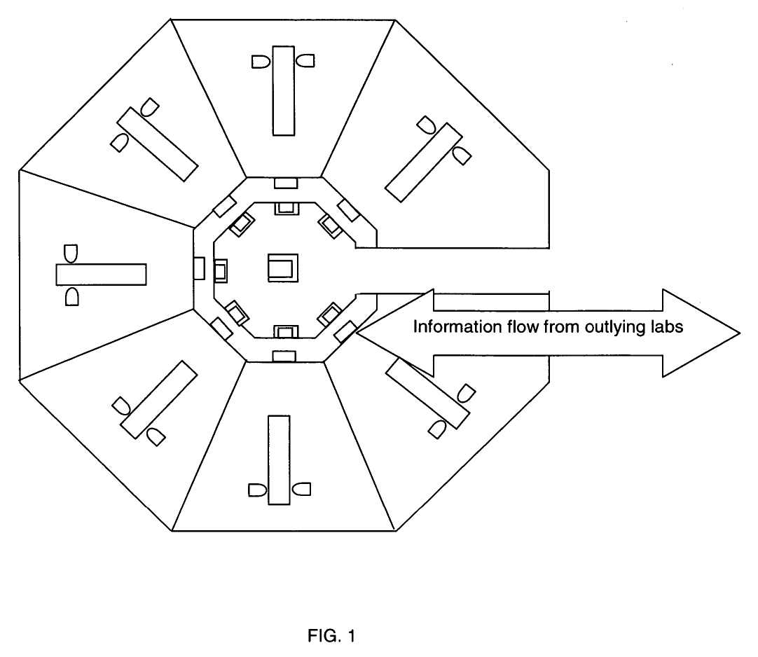

FIG. 2 is an illustration of one embodiment of a system having one or more remote operator stations in communication with a local router;

FIG. 3 is an illustration of one embodiment of a system having one or more visitor operator stations in communication with a full operator station;

FIG. 4 is an illustration of one embodiment of a system having one or more passive operator stations in communication with a full operator station;

FIG. 5 is an illustration of one embodiment of a system having one or more active operator stations in communication with a full operator station;

FIG. 6 is an illustration of one embodiment of a system having one or more other full operator stations in communication with a full operator station;

FIG. 7 is an illustration of a private local network in communication with one or more full operator stations; and

FIG. 8 is an illustration of one embodiment of a system having a satellite communication link for enabling remotely performing a medical procedure at distant locations.

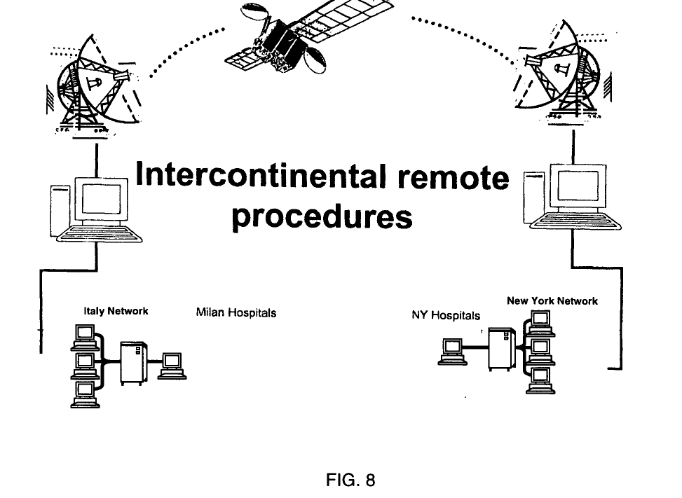

### DETAILED DESCRIPTION OF THE EMBODIMENTS

The following description of the various embodiments is merely exemplary in nature and is in no way intended to limit the invention, its application, or uses.

The network and system for remotely performing minimally invasive procedures comprises a navigation system for controlling the orientation of a medical device such as a catheter within a patient's body. Navigation systems have been commercially developed recently for actuation of medical devices to be steered within a patient's anatomy, from a remote location nearby the patient. An example is the Niobe magnetic navigation system developed and sold by Stereotaxis, Inc. Such a system typically allows for control of the navigation of a minimally interventional device that is inserted within a patient, with the help of a Graphical User Interface and user input devices such as a mouse, keyboard, or joystick that may be located in a control area near the patient.

The concept of remotely mapping cardiac substrates and remotely delivering therapies to the diseased heart has been recently developed with the advent of Stereotaxis Navigational Systems. Physicians possessing expertise in such navigation systems have performed electrophysiology (EP) mapping of heart tissue, and ablation of supraventricular and ventricular tachyarrhythmias. Moreover, given the special nature of the learning curve of procedures using such medical device navigation systems, there is also the utility of remote learning of EP procedures. In one aspect of the present invention, one embodiment of an integrated network system provides for remotely performing minimally invasive medical procedures on a subject body, remotely delivering or performing treatment of a subject body, and remotely providing instruction for learning the procedures being performed by utilizing a satellite-based telecommunication network and/or a fiber-optic communication network. Expert surgeons can perform medical procedures at a full surgical station with a Stereotaxis Navigation system, which other surgeons in remote locations may monitor or even participate in from a passive station under the supervision of the expert surgeon. Alternatively, an expert surgeon may supervise or even perform a medical procedure being conducted at a full surgical station from a remote passive station, while other surgeons at the full surgical station can watch or assist the expert during the procedure. Passive stations may also be used to rehearse a medical procedure at a remote passive or active station, by using pre-operative images of the subject's body presented on the display console. The surgeon can become familiar with the procedure to be performed, and even practice the procedure in a virtual surgery. In this manner, a surgeon may reliably perform a medical procedure on a patient using a minimally interventional device, such as an electrophysiology catheter, from a remote location using the network and system of the present invention.

Various embodiments of the present invention provide for networking one or more Medical Device Navigational Control Systems used in the fields of cardiac mapping and ablation for SVT and VT and in the CRT applications, to provide for remotely performing electrophysiology mapping of a heart, remotely delivering or performing treatment of a subject body, and remotely providing instruction for learning the procedures. In various embodiments of an integrated network of Medical Device Navigational Control systems, one or more features may be provided, including remotely viewing procedures for training purposes, remotely performing procedures with limited passive control of a System, remotely performing procedures with active control of a system, and Full Control systems that allow either passive or active performing of procedures from a remote location. The system provides for performing remote procedures using Stereotaxis navigation equipment and an integrated network utilizing fiber-optic and satellite communication, for learning and remotely conducting EP procedures including ablation of supraventricular and ventricular tachyarrhythmias and for deliver LV stimulation in the CRT setting. Within the system and network, different kinds of operator stations for remote procedures may be provided as detailed below.

Visitor Station. A visitor station will be equipped with a Navigation system console screen and a selection monitor to connect with other active, passive or full stations to enable remote learning about remotely performed medical procedures. In this way, regional teaching centers could be developed in which to organize teaching sessions. Similarly, during cardiology international congresses, a Visitor Station could be useful for directly showing EP procedures and doing dedicated courses for educating people.

Passive Station. A passive station will be equipped with a Navigation console compatible with a Stereotaxis Navigation system, i.e. fully equipped for conducting remote medical procedures from different sites, as part of a shared EP lab, for example. This passive station could be used both for performing medical procedures and for learning procedures on an animal model of cardiac disease. A passive station is connected to at least one Active Station, and is preferably connected to numerous active stations. Thus, Passive Stations may be utilized for advanced remote learning on animal models and for remotely performing medical procedures on patients at Full Surgical Stations. Moreover, Passive Stations can further include a safety algorithm to ensure patient safety. For example, the algorithm may provide predefined zones in which ablation is excluded (i.e. PVs, His bundle, RBBB, etc) depending on the type of remote procedure. The algorithm may also predefine RF automatic controls, where RF energy is applied for no more than 30-60 sec depending on the type of procedure. The algorithm may further provide automatic Impedance monitoring, automatic signal abatement monitoring, and a one-touch safety key.

Active Station. An active station will be equipped with a Navigation console screen and CardioDrive for remote procedures from that site. Many Active Stations could be connected to the same shared Passive Station. In this way, regional centers with a Full Surgical Station with a Stereotaxis Navigation system can be set up and remotely used from many different local Active Stations.

Full Surgical Station. A full working station with Navigation console screen, Cardiodrive and Stereotaxis Navigation system for incoming and outcoming remote procedures can be installed in few high-trained centers. The Full Stations enable incoming operator-assisted remote procedures from other Passive Stations, or outcoming procedures towards other Active Stations requiring consulting and supervision, and intensive learning towards many Visitor Stations.

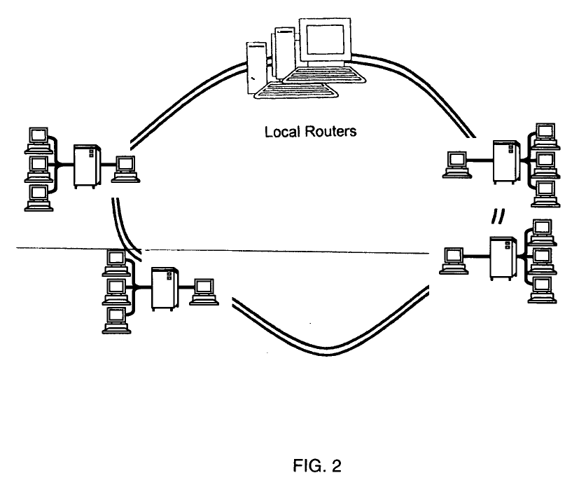

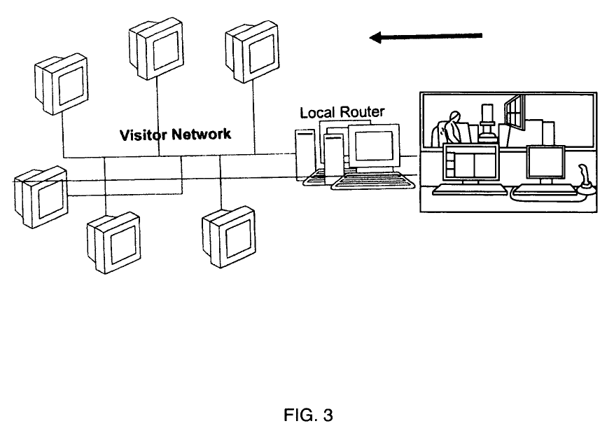

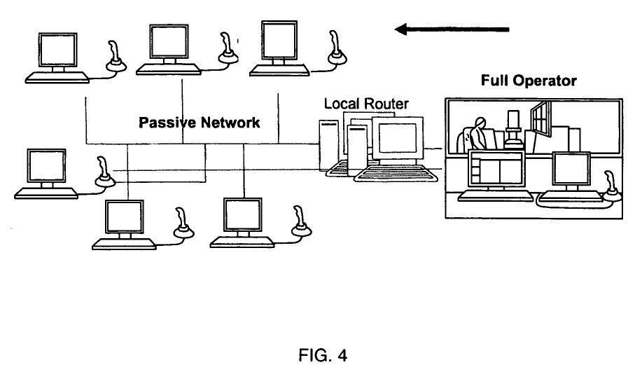

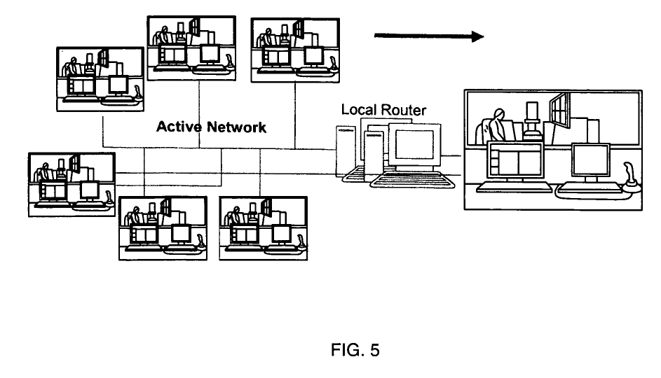

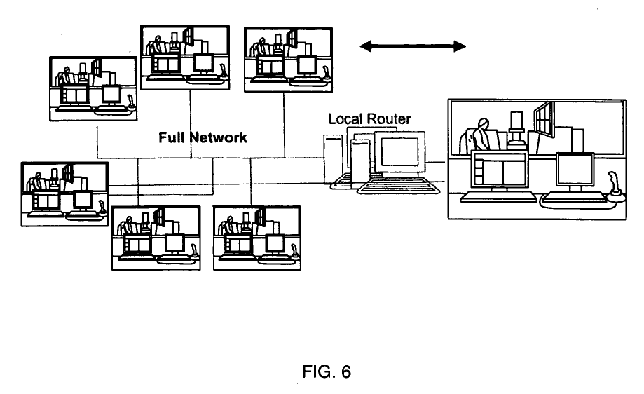

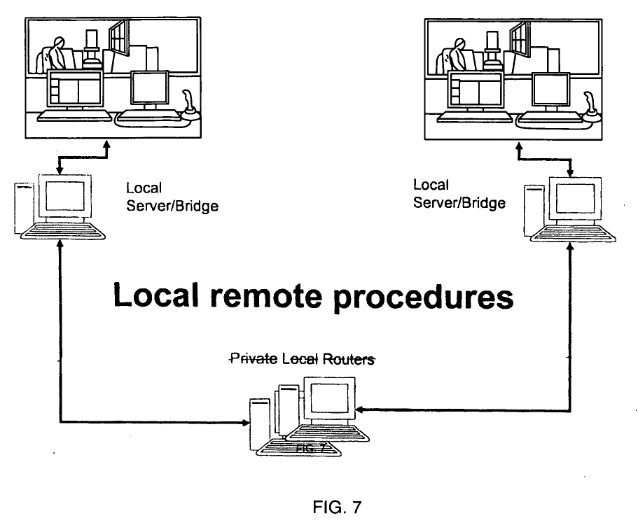

In one embodiment, a system is provided that comprises a local router that may be connected to one or more remote visitor, passive, active or full operator stations as shown in FIG. 2. The local router may be a double ring (active and idle ring) network in communication with servers at remote locations that have joined or connected to the local router. The local router is capable of acquiring the address of the remote operator location, and determining the operator type. For example, FIG. 3 shows a local router that is in communication with a plurality of remote Visitor operator stations, from which students or physicians may monitor or learn about a procedure being performed at a Full operator station via the system and network. Likewise, FIG. 4 shows a local router that is in communication with a plurality of remote Passive operator stations, from which physicians may watch or participate in a limited manner in a procedure being performed at a Full operator station. FIG. 5 shows a local router that is in communication with a plurality of Active operator stations and a Full operator station. From the Full operator station, a physician possessing expertise with such navigation systems can monitor several procedures being performed remotely at several Active operator stations. If an expert physician at the Full operator station determines that a certain remote procedure needs his assistance, the expert physician may use interface means at the Full operator station to control the navigation system at a remote Active operator station, and override the physician at the remote Active operator station. Thus, each patient at each remote Active operator station can receive the benefit of an expert physician supervising the medical procedure being performed. FIG. 6 shows a local router that is in communication with a plurality of Full operator stations and a central Full operator station. Such a network could also be implemented as a private network through a private local router and a plurality of Full operator stations as shown in FIG. 7.

The system further comprises a communication link that provides for communicating between the various surgical stations within the network. The communication link may be a physical communication means such as a fiber-optic communication channel, or alternatively may be a wireless communication means utilizing satellite communication for enabling surgeons to perform procedures from half way around the globe.

For enabling communication from the different sites in which to install different kind of workstations the best technologies to be used are optical fibers on a local basis and satellite connection on an international and intercontinental basis. On a local basis, a server should be installed in each site and a router directly interconnected with each local server. In this way a private and secure network can be set up to enable connections between sites. The communication links between sites optimally comprise fiber optic connection means. Among advantages of using optical fibers, the system achieves the greatest broadcast due to a reduced wavelength, signal frequency 1000 times more than satellite connections (speed *1000), and the highest C*P product (c, capacity of the system; p, repetition pass). Fiber optic communication provides up to 800Gb/sec/km as compared to 10 and 1 Gb/sec/km for radio-based and coaxial wire-based connections,. Fiber-optic connections also provide the lowest attenuation of signal (0.4 dB/km), and allow for direct connections at great distances with a limited number of intermediate signal regenerators and immunity from electromagnetic interferences and safety from fulguration.

In some embodiments, a system is provided for enabling remote monitoring of a medical procedure being performed in a patient's body. The system comprises at least one full operator station having a navigation control system for controlling the orientation of a minimally interventional medical device that is to be guided within a subject body's anatomy, and one or more remote operator stations in communication with the at least one full operator station, wherein the medical procedure may be monitored from the one or more remote operator stations. The remote operator station may be a visitor operator station, from which a medical procedure may be monitored by a student or physician for providing education or training. The remote operator station may be a passive operator station, from which an operator may remotely participate in a limited capacity in a medical procedure being performed at a remote location. The remote operator station may be an active operator station, from which the operator may actively control the medical procedure that is to be performed at a remote location. The remote operator station may also be another full operator station. The network of remote operator stations are in communication with the at least one full operator station via a communication link and a local router. The communication link is preferably a fiber-optic communication means, but may alternatively be a wireless satellite communication link for enabling remote monitoring of a medical procedure that is being performed at a location that is at least part way around the earth.

In another aspect of the present invention, a system is provided for enabling an operator to remotely perform a medical procedure in a patient's body at a remote location. The system comprises at least one full operator station having a navigation control system for controlling the orientation of a minimally interventional medical device that is to be guided within a subject body's anatomy, one or more remote operator stations in communication with the at least one full operator station, wherein the medical procedure may be controlled at least partially by an operator at the one or more remote operator stations. The one or more remote operator stations may be passive operator stations, from which an operator may remotely participate in a limited capacity in a medical procedure being performed at a remote location. The one or more remote operator stations may be active operator stations, from which an operator may actively control the medical procedure that is to be performed at a remote location. The remote operator station may also be another full operator station. Accordingly, a system and network of operator stations may be provided that provide for educational training, hands on training through remotely performing procedures in a limited capacity, and full control of a medical procedure from a remote location that may be a great distance from the patient and medical facility where the procedure is being conducted.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A system for enabling remote monitoring of a medical procedure being performed in a patient's body, the system comprising:
at least one full operator station having a navigation control system for controlling the orientation of a minimally interventional medical device that is to be guided within a subject body's anatomy; and
one or more remote operator stations in communication with the at least one full operator station, wherein the medical procedure may be monitored from the one or more remote operator stations.

2. The system of claim 1 wherein the one or more remote operator stations are visitor operator stations from which a medical procedure is monitored by a student or physician for the purpose of providing education or training.

3. The system of claim 1 wherein the one or more remote operator stations are passive operator stations from which an operator is capable of remotely participating in a limited capacity in a medical procedure being performed at the at least one full operator station.

4. The system of claim 1 wherein the one or more remote operator stations are active operator stations from which an operator is capable of actively controlling the orientation of a minimally interventional medical device that is to be guided within a subject's body at the at least one full operator station location.

5. The system of claim 1, wherein the one or more remote operator stations are other full operator stations from which an operator is capable of actively controlling the orientation of a minimally interventional medical device that is to be guided within a subject's body at the at least one full operator station location.

6. The system of claim 1 wherein the network of remote operator stations are in communication with the at least one full operator station via a communication link.

7. The system of claim 6 wherein the communication link is a fiber-optic communication link.

8. The system of claim 6 wherein the communication link is a fiber-wireless satellite communication link.

9. The system of claim 8, wherein the wireless satellite communication link enables remote monitoring of a medical procedure that is being performed at a location that is at least part way around the earth.

10. The system of claim 6 wherein the network comprises a private local router, and the one or more remote operator stations are full operator stations.

11. A system for enabling an operator to remotely perform a medical procedure in a patient's body at a remote location, the system comprising:
at least one full operator station having a navigation control system for controlling the orientation of a minimally interventional medical device that is to be guided within a subject body's anatomy;
a router for providing communication with the at least one full operator station; and
one or more remote operator stations in communication with the at least one full operator station, wherein an operator at the one or more remote operator stations is capable of at least partially participating in the medical procedure that is being conducted at the at least one full operator station at a remote location.

12. The system of claim 11 wherein the one or more remote operator stations are passive operator stations, from which an operator is capable of at least partially controlling a medical procedure being performed at the at least one full operator station at a remote location.

13. The system of claim 12 wherein the operator is only capable of participating in the medical procedure in a limited capacity.

14. The system of claim 13 wherein the system further comprises an algorithm that includes predetermined constraints on the operator's ability to control a remotely performed medical procedure.

15. The system of claim 11 wherein the one or more remote operator stations may be active operator stations, from which an operator is capable of actively controlling the medical procedure that is to be performed at the at least one full operator station at a remote location.

16. The system of claim 11 wherein the one or more remote operator stations are other full operator stations from which an operator is capable of actively controlling the orientation of a minimally interventional medical device that is to be guided within a subject's body at the at least one full operator station location.

17. The system of claim 11 wherein the network of remote operator stations are in communication with the at least one full operator station via a communication link.

18. The system of claim 17 wherein the communication link is a fiber-optic communication link.

19. The system of claim 17 wherein the communication link is a fiber-wireless satellite communication link.

20. The system of claim 19, wherein the wireless satellite communication link enables remote monitoring of a medical procedure that is being performed at a location that is at least part way around the earth.
